(19) United States
(12) Patent Application Publication
(10) Pub. No.: US 2007/0128124 A1
(43) Pub. Date: Jun. 7, 2007

(54) Aerosolized capreomycin for inhibition of pulmonary tuberculosis

(76) Inventors: Carl N. Kraus, Gaithersburg, MD (US); Clifton E. Barry III, Rockville, MD (US); Bernardan T. Doan, Falls Church, VA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/527,557

(22) PCT Filed: Sep. 12, 2003
(86) PCT No.: PCT/US03/28889

§ 371 Date: Nov. 25, 2005

Related U.S. Application Data
(60) Provisional application No. 60/410,601, filed on Sep. 12, 2002.

(51) Int. Cl. A61K 9/14; A61K 38/12

(52) U.S. Cl. 424/46; 514/9

(57) ABSTRACT

The invention provides systems, methods, and compositions for the aerosolized administration of capreomycin to persons in need thereof. Administration of capreomycin by aerosol can be used to reduce the infectivity of persons with tuberculosis, or to reduce the severity or duration of a tuberculosis infection. The invention further provides for the use of capreomycin for the manufacture of a medicament for aerosolized administration to a person in need thereof.

### CROSS-REFERENCES TO RELATED APPLICATIONS

This application claims priority to U.S. Provisional Application No. 60/410,601, filed Sep. 12, 2002, and U.S. Provisional Application No. 60/497,892, filed Aug. 25, 2003. The contents of both of these applications are hereby incorporated by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE INVENTION

This invention relates to the inhibition of pulmonary tuberculosis.

### BACKGROUND OF THE INVENTION

The World Health Organization (“WHO”) estimates that as much as one-third of the world's population is infected with tuberculosis. In 1998, Mycobacterium tuberculosis (“MTB” or “M. tuberculosis”) was estimated to have infected 7.25 million people and resulted in 2.9 million fatalities (Farrner, P. et al., Int J Tuberc Lung Dis 2:869 (1998)). Underlying these statistics is an emerging epidemic of multiple drug-resistant (“MDR”) tuberculosis that severely undermines control efforts and is transmitted indiscriminately across national borders (Viskum, K. et al., Int J Tuberc Lung Dis 1:299 (1997); Bass, J. B. et al., Am J Respir Crit Care Med 149:1359 (1994)). Resistance to any of the front-line drugs generally bodes poorly for the patient, who then is committed to a regimen of less active “second-line” therapies. Where multidrug resistance is suspected, the WHO recommends that three or more drugs be administered concomitantly, to decrease the chance that the organism will be able to develop resistance to all of the agents. The currently recommended “intensive phase” drug regimen is composed of isoniazid, rifampin, pyrazinamide, and ethambutol (streptomycin can be substituted for ethambutol in the regimen). Typically, the drugs are taken daily for eight weeks, after which the isoniazid and rifampin are taken two to three times a week for the “continuation phase” of therapy, which lasts for sixteen weeks, for a total of at least six months of therapy.

Capreomycin is a cyclic polypeptide antibiotic initially isolated from Streptomyces capreolus by Herr in 1961 at Lilly Research Laboratories. Capreomycin, available commercially under the name Capastat®, has significant in vitro and in vivo efficacy against M. tuberculosis, M. bovis, M. kansasii, and M. avium. Against MTB, it has a minimum inhibitory concentration (MIC) of 2.5 mcg/ml. Human pharmacokinetics are favorable in that the maximum serum concentration (Cmax) that is reached two hours after intramuscular injection is 25.0 mcg/ml, approximately 10 times the MIC, insuring adequate serum concentrations for mycobactericidal activity to occur. Capreomycin is not a front line agent for the treatment of pulmonary tuberculosis, however, due to its mode of administration and toxicity profile. Capreomycin must be given parenterally either by deep intramuscular injection or by intravenous injection as oral absorption is quite poor. Toxicities of capreomycin mirror many of the aminoglycosides, including renal and ototoxicity, with dose reductions required for compromised creatinine clearance. These toxicities can occur in up to 10% of patients. Capreomycin has since been reserved as a second line agent for use in drug-resistant cases of tuberculosis.

It would desirable to be able to reduce the toxicities associated with capreomycin use so that its utility as an anti-tubercular agent can be extended.

### BRIEF SUMMARY OF THE INVENTION

This invention provides systems and compositions for the aerosolized administration of capreomycin to persons in need thereof.

In one group of embodiments, the invention provides systems comprising capreomycin and a device for introducing said capreomycin into gases for inhalation by a person in need thereof. The capreomycin may be introduced into the gases as, for example, a solution, a suspension, a powder, or a spray. In some embodiments, the device can be a nebulizer, a metered dose inhaler, or a dry powder inhaler. In embodiments where the device is a nebulizer, the nebulizer can be, for example, a heated nebulizer, an ultrasonic nebulizer, a gas nebulizer, a venturi nebulizer, or a refillable nebulizer. The capreomycin can be introduced into the gases in an average particle size of between 1 and 10 microns, and can have an average particle size of between 2 and 6 microns or an average particle size of about 3 to about 5 microns. The capreomycin can be provided as a powder.

In another group of embodiments, the invention provides formulation of capreomycin suitable for aerosol inhalation. The capreomycin of such formulations can have an average particle size between 1 and 10 microns. The capreomycin can be complexed or associated with a polysaccharide.

The invention further provides methods of inhibiting the growth of Mycobacterium tuberculosis (“MTB”). The method comprises introducing capreomycin into gases to be inhaled by a patient in need thereof. The capreomycin can be introduced into the gases as a solution, a suspension, a powder, or a spray. The capreomycin can be introduced into the gases in an average particle size of between 1 and 10 microns, and can be complexed or associated with a polysaccharide. The capreomycin can be introduced into the gases by a nebulizer, a metered dose inhaler, or a dry powder inhaler. Where the capreomycin is introduced into the gases by a nebulizer, the nebulizer can be, for example, a heated nebulizer, an ultrasonic nebulizer, a gas nebulizer, a venturi nebulizer, or a refillable nebulizer.

The invention further provides methods of inhibiting the growth of Mycobacterium tuberculosis (“MTB”) in a patient. The method comprises administering to a lung of the patient an aerosol formulation of capreomycin, wherein said capreomycin inhibits the growth of MTB in the patient. Typically, the capreomycin is administered to said lung as a solution, a suspension, a powder, or a spray. The capreomycin may be administered to the lung by a nebulizer, a metered dose inhaler, or a dry powder inhaler. Where the capreomycin is administered to the lung by a nebulizer, the nebulizer can be, for example, a heated nebulizer, an ultrasonic nebulizer, a gas nebulizer, a venturi nebulizer, or a refillable nebulizer.

The invention further provides methods of reducing infectivity of a person infected with Mycobacterium tuberculosis (“MTB”). The method comprises administering to a lung of said person an aerosol formulation of capreomycin, wherein said capreomycin reduces the infectivity of said person. The capreomycin may be administered to the lung as a solution, a suspension, a powder, or a spray. The capreomycin may be administered to the lung by a nebulizer, a metered dose inhaler, or a dry powder inhaler. Where the capreomycin is administered to the lung by a nebulizer, the nebulizer can be, for example, a heated nebulizer, an ultrasonic nebulizer, a gas nebulizer, a venturi nebulizer, or a refillable nebulizer.

The invention further provides use of capreomycin for manufacture of a medicament for aerosolized administration to a lung as a solution, a suspension, a powder, or a spray. The medicament may be suitable for administration by, for example, a nebulizer, a metered dose inhaler, or a dry powder inhaler.

In yet another embodiment, the invention provides formulations of lyophilized capreomycin having an average particle size of from about 1 to about 10 microns.

### BRIEF DESCRIPTION OF THE DRAWINGS

FIG. 1 shows the efficacy of the antituberculars isoniazid, capreomycin, and rifampicin at 10 times minimum inhibitory concentration (“MIC”) in log phase cultures of M. tuberculosis. The X axis states the time at which readings were taken, while the Y axis shows the log of the relative light units (“RLU”).

FIGS. 2A and 2B show the efficacy of the antituberculars isoniazid, capreomycin, and rifampicin at 10 times minimum inhibitory concentration (“MIC”) against stationary phase cultures of M. tuberculosis. FIG. 2A. The Y axis shows the log of the relative light units (“RLU”). The X axis shows the time after the antitubercular was added. FIG. 2B. Colony forming units (“CFUs”) were plated and assayed for correlation to the RLUs shown in FIG. 2A.

FIG. 3 shows an exemplar apparatus for in vivo administration of aerosol to an animal subject.

FIGS. 4A and 4B show the in vivo efficacy of aerosolized isoniazid and rifampicin in C57/BL6 mice in an aerogenic model of murine tuberculosis. FIG. 4A. Lungs. FIG. 4B. Spleen. Both Figures: Y axis: Log of colony forming units (“CFUs”).

FIGS. 5A and 5B show the in vivo efficacy of aerosolized capreomycin in C57/BL6 mice in an aerogenic model of murine tuberculosis. FIG. 5A. Lungs. FIG. 5B. Spleen. Both Figures: Y axis: Log of colony forming units (“CFUs”).

### DETAILED DESCRIPTION

Introduction

Capreomycin was discovered in the 1960s, and has been used against tuberculosis for over 30 years. It has always been administered by intramuscular injection or intravenous administration because, as a cyclic peptide, it was not sufficiently available when ingested.

Surprisingly, it has now been discovered that capreomycin is active against tuberculosis when administered by inhalation into the lungs. This could not have been predicted. First, due to the complexities and interactions between different types of compounds and the cells of the lung, it could not be predicted from results with other drugs whether capreomycin would successfully cross from inspired air into the cells composing the alveoli of the lungs. Second, researchers studying other drugs currently in advanced clinical trials as inhalation therapies, such as insulin and pain killers, have found that those drugs rapidly cross the thin layer of cells composing the alveoli pass into the systemic circulation. The ability to deliver an adequate therapeutic dose of a therapeutic aerosol therefore depends in part on the physical characteristics of the aerosol. These characteristics include the particle diameter, electrical charge, and density.

Other drugs have been administered to achieve localized therapy in the lung for lung disorders. Tuberculosis is characterized, however, in part by the formation of granulomas, in which the bacteria enter periods of relative inactivity and are protected by a thick layer of epitheloid cells. Whereas some conditions of the lung may be treated by delivery of an agent to the bronchi of bronchioles, treatment of TB requires delivery to the alveoli, which have a tremendous absorptive, thin surface area, favoring delivery of the agent into the systemic circulation, reducing the advantage of delivering the agent to the lung in the first instance.

It is therefore not possible to predict from results with any particular agent whether administration of another, unrelated agent to a patient will result in levels of the agent sufficient to kill relatively inactive TB bacteria and prevent recurrences of the disease. Thus, for tuberculosis agents, in particular, it is desirable to have a sufficiently high residence time of the agent in the lung to provide adequate dosing to the granuloma. Once the therapeutic molecule has reached the alveoli (and, if a powder, has solubilized in the mucus), whether the molecule remains in the lung or undergoes pulmonary absorption depends on additional factors. For example, small molecules can solubilize by diffusion or by carrier-mediated transport, while large molecules may undergo bulk transport through large pores or pinocytosis (reviewed in Suarez and Hickey, “Drug Properties Affecting Aerosol Behavior”, Respir Care 45(6):652-666 (2000) (hereafter, “Suarez and Hickey”). Suarez and Hickey further note that drug residence time depends on both clearance and absorption times, which are governed by what they characterize as the physiochemical properties of the drug, including the drug's molecular weight, dissolution rate, partition coefficient, and charge. Therefore, for local, as opposed to systemic delivery, it is desirable to have slow clearance and absorption of the agent from the lung.

Given the confluence of factors, it could not be predicted, prior to the present discoveries, whether aerosol administration of capreomycin would achieve therapeutic levels in the lung or whether the drug would pass into the systemic circulation too rapidly for therapeutic concentrations of the drug to develop. Further, it could not be predicted whether such a local concentration in the lung would be accompanied by development of high systemic levels that would possibly result the undesirable side effects associated with systemic administration.

It has now been discovered that inhalation administration of capreomycin permits the development of therapeutically useful levels in the lung. It is further believed that the risk-benefit ratio of the drug is thereby improved, by allowing therapeutically effective levels to be reached in the lung before high systemic levels of the drug are reached, increasing the patient population that can benefit from its use. Moreover, the administration of the drug by inhalation performed by the patient, optionally under the supervision of personnel who themselves have undergone only brief training, whereas the alternative methods of administration, intramuscular injection or intravenous infusion, require personnel who have been trained in administering injections and introduces the risk of needle-stick accidents and consequent infection. Since a large percentage of tuberculosis cases occur in some of the poorest countries in the world, reducing the degree of training needed to administer the drug is a significant advantage of the invention. While the studies herein were performed in mice, given the similarity of mammalian lungs, it is expected that the results found in the studies performed herein are predictive of the results in other mammals, including man.

Capreomycin is usually provided in a lyophilized form, which must be reconstituted with sterile saline before injection. In less developed countries, it is desirable to avoid the need for reconstituting the drug with sterile solutions. In one embodiment of the present invention, the capreomycin is provided in lyophilized single unit doses and is administered by inhalation as a powder. The powdered drug is then dissolved in the mucus lining the lung and therefore does not need to be reconstituted before administration. Thus, while the invention will prove useful in industrialized countries, it offers particular advantages in less developed countries.

Inhalation capreomycin can be administered for various purposes. Most commonly, it can be administered to persons who have contracted multi-drug resistant (“MDR”) tuberculosis (“TB”), that is, tuberculosis that is resistant to both of the front line agents, isoniazid or rifampin. Often, MDR TB arises when a patient has not followed the full course of prescribed therapy. In these cases, the administration of capreomycin is intended in part to reduce the growth of MTB in the patient. Similarly, inhalation capreomycin can be administered as a front line therapy. Persons of skill will also recognize that one of the problems with active infections with MTB is the propensity to infect others in close proximity to sputum positive for the presence of TB organisms. Inhalation capreomycin can also be administered to reduce the growth of MTB in the sputum, thereby reducing the infectivity of patients whose sputum is positive for the presence of MTB. This would therefore serve not only as a therapeutic benefit to the individual patient, but may directly impact public health by decreasing the number of people exposed to, and subsequently infected by an “index case” of tuberculosis in the community (an index case is the first case that brings a disease to the attention of medical personnel). The results of the studies reported herein indicate that inhaled capreomycin can also be effective against “persisters,” that is, MTB organisms that remain in a state of limited growth and metabolic quiescence for an extended time before resuming an active growth state.

When used for inhalation therapy, capreomycin can be administered in either a liquid aerosol, typically in a nebulizer, or in the form of a powder. In aerosol formulations, the drug may be provided as a powder, which is reconstituted at the point of administration or as a sterile liquid in which the drug is already mixed to a desired concentration. Conveniently, the drug may be provided in a package intended to provide a single dose, although packaging providing multiple doses may also be used. Powders may likewise be used in a device that permits multiple administrations from a reservoir of the powder, or may be provided in a package containing a single dose.

The invention includes formulations of the drug for inhalation. The powder formulations will comprise the drug in an average particle size suitable for inspiration. It is desirable if the particles are of an average size and weight such that they can reach the alveoli. It is known in the art that particles that are too large will primarily land in the mouth and throat before reaching the alveoli. On the other hand, it is also known in the art that the particles cannot be so small or so light that once they reach the deep lung, they are simply expelled in exhalation rather than deposited in the lung. Preferably, the particles have an average size of between 0.1 and 20 microns and more preferably have an average size between 0.5 and 10 microns. For some powder uses, it is desirable for the mass median aerodynamic diameter (“MMAD”) to be between 5 and 10 microns, and preferably about 6-7 microns. For nebulizer use, it may be desirable for the particles to have a somewhat smaller MMAD of between about 1-5 microns. Such particles can conveniently be formed by use of jet or ultrasonic nebulizers.

Preparation of powders for inhalation administration is well known in the art, as taught in, for example, U.S. Pat. No. 6,309,671. Optionally, the drug may be stabilized during the drying process by the use of one or more polyol, such as a carbohydrate or a polysaccharide, as taught in, e.g., U.S. Pat. Nos. 6,290,991 and 4,891,319 and International Publication WO 95/33488, or a monosaccharide sugar alcohol, as taught in International Publication WO 99/47174 (which also teaches use of calcium lactate). Trehalose is particularly used for this purpose, as taught in, e.g., International Publication WO 97/28788. Dextran, mannose, or mannitol may also be used, alone or in combination with trehalose. See, e.g, Hanyu et al., EP Application 1 136 068 A2 (stabilizing protein by use of mannitol prior to drying to form powder of protein for inhalation). For purposed of this application, the presence of polyols, polysaccharides, and monosaccharide sugar alcohols and the like to stabilize a protein drugs such as capreomycin during drying is described as having these agents complexed or associated with the capreomycin. The particles formed may be solid or hollow, and may be porous. The formation of porous particles of a size appropriate for use in inhalation is described in, for example, European patent 432232.

Definitions

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

Capreomycin is a polypeptide antibiotic derived from Streptomyces capreolus originally isolated by Eli Lilly & Co. in 1961. The drug is a complex of 4 anti-microbiologically active components, capreomycin IA, IB, IIA, and IIB. For example, IA and IB differ only in that an R group off of one carbon is a hydroxyl in IA and a hydrogen in IB. Capreomycin has been known for decades to have activity against tuberculosis, see generally, JAMA, 223:179-80 (1973), and is currently used as a second line agent against multidrug resistant tuberculosis (MDR-TB). The drug is manufactured commercially as the disulfate salt under the name Capastat® sulfate by Eli Lilly & Co. (Indianapolis, Ind.). References to capreomycin herein include pharmaceutically acceptable salts thereof unless otherwise required by context, and can refer either to the complex of 4 active components known in the art or to a anti-microbially active component thereof, such as capreomycin IA, unless otherwise required by context.

“Aerosolized” or “aerosolizable” particles are particles which, when dispensed into a gas stream by either a passive or an active inhalation device, remain suspended in the gas for an amount of time sufficient for at least a portion of the particles to be inhaled by the patient, so that a portion of the particles reaches the lungs. In the art, the term aerosol usually designates a substance that is dissolved in or mixed with a liquid, as opposed to a dry powder, which may also be mixed into a gas stream for inhalation.

“Dry powder” refers to a powder composition that typically contains less than about 20% moisture, preferably less than 10% moisture, can contain less than about 5% moisture, and most preferably contains less than about 3% moisture, depending upon the particular formulation.

A dry powder that is “suitable for pulmonary delivery” refers to a composition comprising solid (i.e., non-liquid) or partially solid particles that are capable of being (i) readily dispersed in/by an inhalation device and (ii) inhaled by a subject so that a portion of the particles reach the lungs to permit penetration into the alveoli. Such a powder is considered to be “respirable”.

A “dry powder inhaler” or “DPI” is an inhaler that provides a dry powder suitable for pulmonary delivery in which the powder is driven by patient inspiration alone or with power assistance.

The term “inhalation device” refers herein to any device permitting inhalation of therapeutic amounts of capreomycin into the lungs. It can refer individually or collectively, for example, to dry powder inhalers, metered dose inhalers, or nebulizers.

A “metered dose inhaler”, or “MDI”, is a device that provides a propellant-based inhalation or nasal aerosol. Such devices typically have a therapeutically active substance dissolved or suspended in a propellant or a mixture of solvents, propellants and other excipients.

Mass median diameter” or “MMD” is a measure of mean particle size, since it is common for powders to generally be polydisperse (i.e., consist of a range of particle sizes). MMD values may conveniently be determined by centrifugal sedimentation, although any number of commonly employed techniques can be used for measuring mean particle size (e.g., electron microscopy, light scattering, laser diffraction).

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by cascade impaction, unless otherwise indicated.

“Nebulizer” is defined by the United States Food and Drug Administration (“FDA”) at 21 C.F.R. § 868.5630 (a) as “a device intended to spray liquids in an aerosol form into gases that are delivered directly to the patient for breathing. Heated, ultrasonic, gas, venturi, and refillable nebulizers are included in this generic type of device.” The devices can rely on patient inspiration alone or in combination with power assistance or on propellant.

“Pharmaceutically acceptable excipient or carrier” refers to an excipient that may optionally be included in the compositions of the invention, and taken into the lungs with no significant adverse toxicological effects to the subject, and particularly to the lungs of the subject.

“Pharmacologically effective amount” or “physiologically effective amount of a bioactive agent” is the amount of an active agent present in an aerosolizable composition as described herein that is needed to provide a desired level of active agent in the bloodstream or at the site of action (e.g., the lungs) of a subject to be treated to give an anticipated physiological response when such composition is administered to the lungs. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein.

Devices for Administering Capreomycin

Devices for delivering drugs to the lungs are well known in the art. The devices typically deliver either an aerosol of an therapeutically active agent in a solution, or a dry powder of the agent. To aid in providing reproducible dosages of the agent, dry powder formulations often include substantial amounts of excipients, such as polysaccharides, as bulking agents.

Detailed information about the delivery of therapeutically active agents in the form of aerosols or as powders is available in the art. Persons of skill are aware, for example, that the Food and Drug Administration (“FDA”) provides a series of Guidance documents which, while not binding on the agency, provide its thinking on aspects of various matters and which are generally followed in the industry. In this regard, the FDA's Center for Drug Evaluation and Research (“CDER”) provides detailed guidance in an official document entitled: Guidance for Industry: Nasal Spray and Inhalation Solution, Suspension, and Spray Drug Products—Chemistry, Manufacturing, and Controls Documentation (Office of Training and Communications, Division of Drug Information, CDER, FDA, July 2002). This guidance is available in written form from CDER, or can be found on-line by entering “http://www.” followed by “fda.gov/cder/guidance/4234fnl.htm”. The FDA has also made detailed draft guidance available on dry powder inhalers and metered dose inhalers. See, Metered Dose Inhaler (MDI) and Dry Powder Inhaler (DPI) Drug Products—Chemistry, Manufacturing, and Controls Documentation, 63 Fed. Reg. 64270, (November 1998).

In some aspects of the invention, the capreomycin is dissolved or suspended in a solvent, e.g., water, ethanol, or saline, and administered by nebulization. By law (21 C.F.R. §200.51) all aqueous-based oral inhalation drug products must be manufactured as sterile products. A nebulizer produces aerosol of fine particles by breaking a fluid into fine droplets and dispersing them into a flowing stream of gas. The droplet size from a medical nebulizer is considerably smaller than a conventional spray atomizer. Medical nebulizers are designed to convert water or aqueous solutions or colloidal suspensions to aerosols of fine, inhalable droplets that can enter the lungs of a patient during inhalation and deposit on the surface of the respiratory airways. Typical pneumatic (compressed gas) medical nebulizers develop approximately 15 to 30 microliters of aerosol per liter of gas in finely divided droplets with volume or mass median diameters in the respirable range of 2 to 4 micrometers. Medication intended for aerosolization generally consists of a solute (the medication) mixed into a base solution in which the solute may be dissolved. Predominantly, water or saline solutions are used with low solute concentrations, typically ranging from 1.0 to 5.0 mg/mL.

Nebulizers for delivering an aerosolized solution to the lungs are commercially available from a number of sources, including the AERx™ (Aradigm Corp., Hayward, Calif.), the Ultravent™ (Mallinkrodt), and the Acorn II®. (Vital Signs Inc., Totowa, N.J.). Typical dosing for nebulizers are set forth in, e.g., Metered dose inhalers are also known and available. Breath actuated inhalers typically contain a pressurized propellant and provide a metered dose automatically when the patient's inspiratory effort either moves a mechanical lever or the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978; and 4,896,832.

Dry Powder Preparation and Administration

Dry powder formulations are preferably prepared by spray drying. Spray drying of the formulations is carried out, for example, as described generally in the “Spray Drying Handbook”, 5th ed., K. Masters, John Wiley & Sons, Inc., N.Y., NY (1991), and in Platz, R., et al., International Patent Publication No. WO 97/41833 (1997).

Capreomycin is soluble in water and can be sprayed dried from an aqueous solution. Typically, the capreomycin is first dissolved in water, optionally containing a physiologically acceptable buffer. The pH range of the solution is generally between about 4 and 11, with a pH nearer to neutral being preferred, since such pHs may aid in maintaining the physiological compatibility of the powder after the powder is dissolved within the lung. The aqueous formulation may optionally contain additional water-miscible solvents, such as acetone, alcohols and the like. Representative alcohols are lower alcohols such as methanol, ethanol, propanol, isopropanol, and the like. The pre-spray dried solutions will generally contain solids dissolved at a concentration from 0.01% (weight/volume) to about 20% (weight/volume), usually from 0.1% to 3% (weight/volume).

The solution is then spray dried in a conventional spray drier, such as those available from commercial suppliers such as Niro A/S (Denmark), or Buchi (Switzerland), resulting in a dispersible, dry powder. The gas used to spray dry the material is typically air, although inert gases such as nitrogen or argon may also be used. Moreover, the temperature of both the inlet and outlet of the gas used to dry the sprayed material is such that it does not cause decomposition of the capreomycin. Such temperatures are typically determined experimentally, although generally, the inlet temperature will range from about 50° C. to about 200° C. while the outlet temperature will range from about 30° C. to about 150° C.

Alternatively, the composition may be prepared by spray-drying a suspension, as described in Gordon, U.S. Pat. No. 5,976,574. In this method, the hydrophobic drug is dissolved in an organic solvent, e.g., methanol, ethanol, isopropanol, acetone, heptane, hexane chloroform, ether, followed by suspension of the hydrophilic excipient in the organic solvent to form a suspension. The suspension is then spray-dried to form particles. Preferred solvents, for both of the above spray-drying methods include alcohols, ethers, ketones, hydrocarbons, polar aprotic solvents, and mixtures thereof.

Dry powders of the invention may also be prepared by combining aqueous solutions or suspensions of the formulation components and spray-drying them simultaneously in a spray-dryer, as described in Gordon, U.S. Pat. No. 6,001,336. Alternatively, powders may be prepared by lyophilization, vacuum drying, spray freeze drying, super critical fluid processing, air drying, or other forms of evaporative drying. In some instances, it may be desirable to provide the dry powder formulation in a form that possesses improved handling/processing characteristics, e.g., reduced static, better flowability, low caking, and the like, by preparing compositions composed of fine particle aggregates, that is, aggregates or agglomerates of the above-described dry powder particles, where the aggregates are readily broken back down to the fine powder components for pulmonary delivery, as described, e.g., in Johnson, U.S. Pat. No. 5,654,007.

In another approach, dry powders may be prepared by agglomerating the powder components, sieving the materials to obtain agglomerates, spheronizing to provide a more spherical agglomerate, and sizing to obtain a uniformly-sized product, as described, e.g., in Ahlneck, International Publication No. WO 95/09616. Dry powders may also be prepared by blending, grinding, sieving or jet milling formulation components in dry powder form.

Once formed, the dry powder compositions are preferably maintained under dry (i.e., relatively low humidity) conditions during manufacture, processing, and storage. Irrespective of the drying process employed, the process will preferably result in respirable, highly dispersible particles comprising capreomycin.

Features of Dry Powder Formulations

Powders of the invention are further characterized by several features, most notably, (i) consistently high dispersivities, which are maintained, even upon storage, (ii) small aerodynamic particles sizes (MMADs), (iii) fine particle dose values, i.e., powders having a high percentage of particles sized less than 3.3 microns MMAD, all of which contribute to the improved ability of the powder to penetrate to the tissues of the lower respiratory tract (i.e., the alveoli) for localized administration. These physical characteristics of the dry powders are useful in maximizing the efficiency of aerosolized delivery of such powders to the deep lung.

Dry powders of the invention are composed of aerosolizable particles effective to penetrate into the lungs. The particles of the invention have a mass median diameter (MMD) of less than about 20 microns, and preferably less than about 10 microns.

The powders of the invention are further characterized by an aerosol particle size distribution preferably less than about 10 microns mass median aerodynamic diameter (MMAD). The powders will generally have a moisture content below about 20% by weight, usually below about 10% by weight, and preferably below about 6% by weight. Such low moisture-containing solids tend to exhibit a greater stability upon packaging and storage.

Administration of Aerosolized Capreomycin

The formulations described herein may be delivered using any suitable dry powder inhaler (DPI), i.e., an inhaler device that utilizes the patient's inhaled breath as a vehicle to transport the dry powder drug to the lungs. Such devices are described in, for example, U.S. Pat. Nos. 5,458,135; 5,740,794; and 5,785,049.

When administered using a device of this type, the powder is contained in a receptacle having a puncturable lid or other access surface, preferably a blister package or cartridge, where the receptacle may contain a single dosage unit or multiple dosage units. Convenient methods for filling large numbers of cavities (i.e., unit dose packages) with metered doses of dry powder medicament are described, e.g., in Parks, International Publication WO 97/41031.

Other dry powder dispersion devices for pulmonary administration of dry powders include those described, for example, in Newell, European Patent No. EP 129985; in Hodson, European Patent No. EP 472598, in Cocozza, European Patent No. EP 467172, and in Lloyd, U.S. Pat. Nos. 5,522,385; 4,668,281; 4,667,668; and 4,805,811. Other suitable devices include dry powder inhalers such as the Rotahaler™ (Glaxo), Discus™ (Glaxo), Spiros™. inhaler (Dura Pharmaceuticals), and the Spinhaler™ (Fisons). Dry powders may also be delivered using a pressurized, metered dose inhaler (MDI), e.g., the Ventolin™ metered dose inhaler, containing a solution or suspension of drug in a pharmaceutically inert liquid propellant, e.g., a chlorofluorocarbon or fluorocarbon, as described in U.S. Pat. Nos. 5,320,094 and 5,672,581.

Prior to use, dry powders are generally stored under ambient conditions, and preferably are stored at temperatures at or below about 25° C., and relative humidities (RH) ranging from about 30 to 60%. More preferred relative humidity conditions, e.g., less than about 30%, may be achieved by the incorporation of a desiccating agent in the secondary packaging of the dosage form.

### EXAMPLES

The following examples are offered to illustrate, but not to limit the claimed invention.

### Example 1

This Example reports in vitro studies comparing capreomycin to two front line anti-tubercular agents.

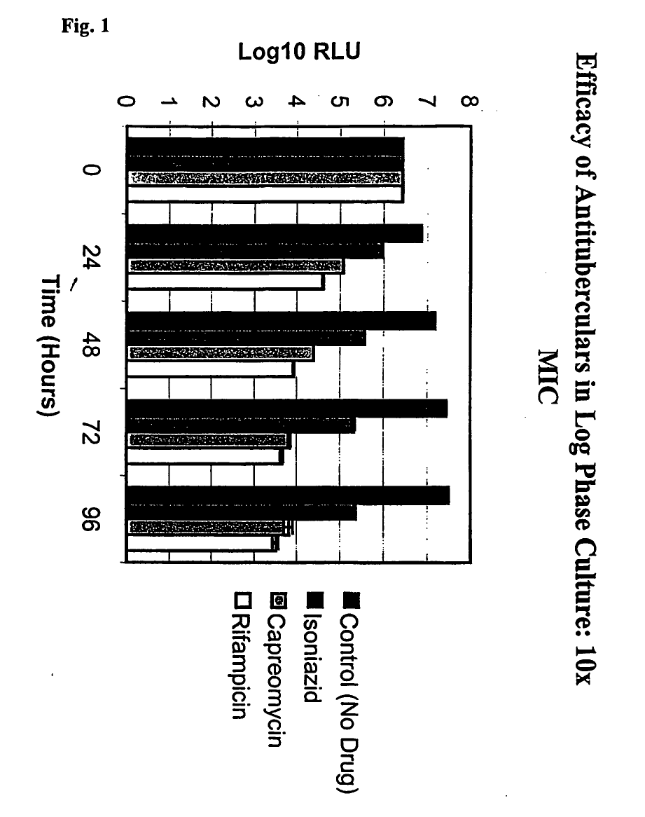

Using a luciferase reporter in MTB strain H37Rv, relative light units (RLU) can be utilized as a surrogate for mycobacterial viability as a means of having real time quantitation for an organism that requires 6 weeks for evaluation of colony forming units (“CFU”). When compared with rifampicin or isoniazid (front line antituberculars) at 10 times the minimum inhibitory concentration, or MIC, the efficacy of capreomycin is superior to that of isoniazid and is as efficacious as rifampicin in log phase growth conditions (FIG. 1). The efficacy of isoniazid using RLUs as an endpoint reaches peak efficacy at 72 hours with an approximate 1.5 log decrease from untreated cultures. Rifampicin reaches peak efficacy at approximately 72 hours with a 3.5 log decrease from untreated cultures and capreomycin has similar mycobactericidal activity with a 3.5 log decrease from untreated controls.

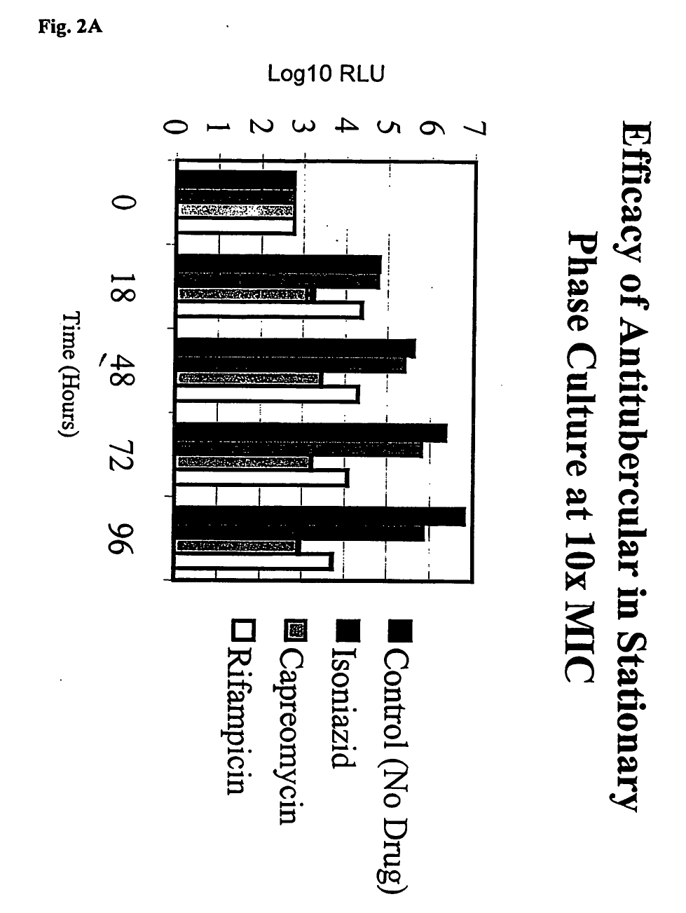

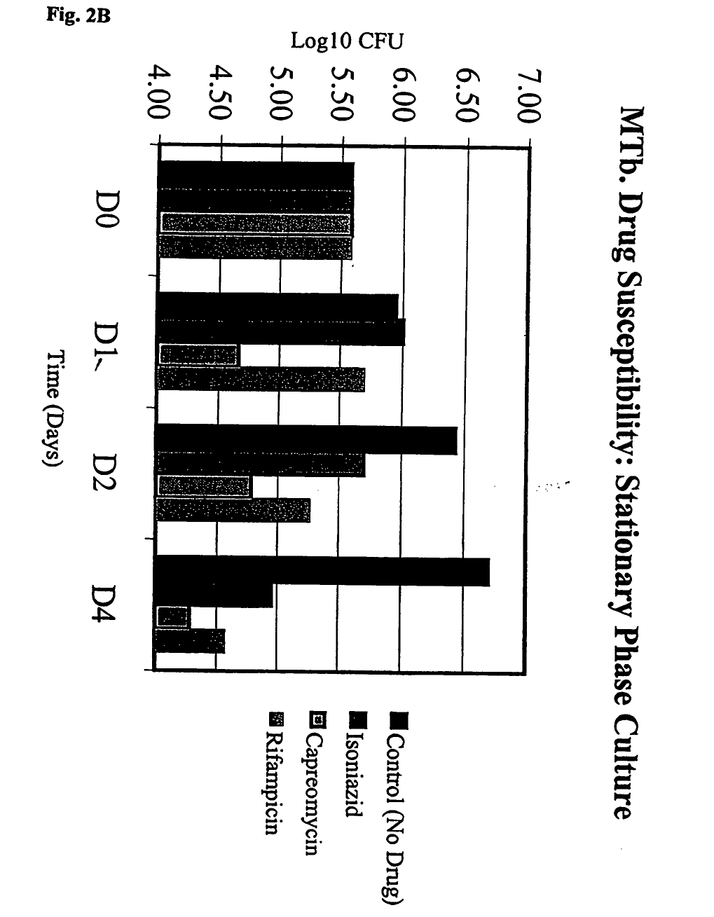

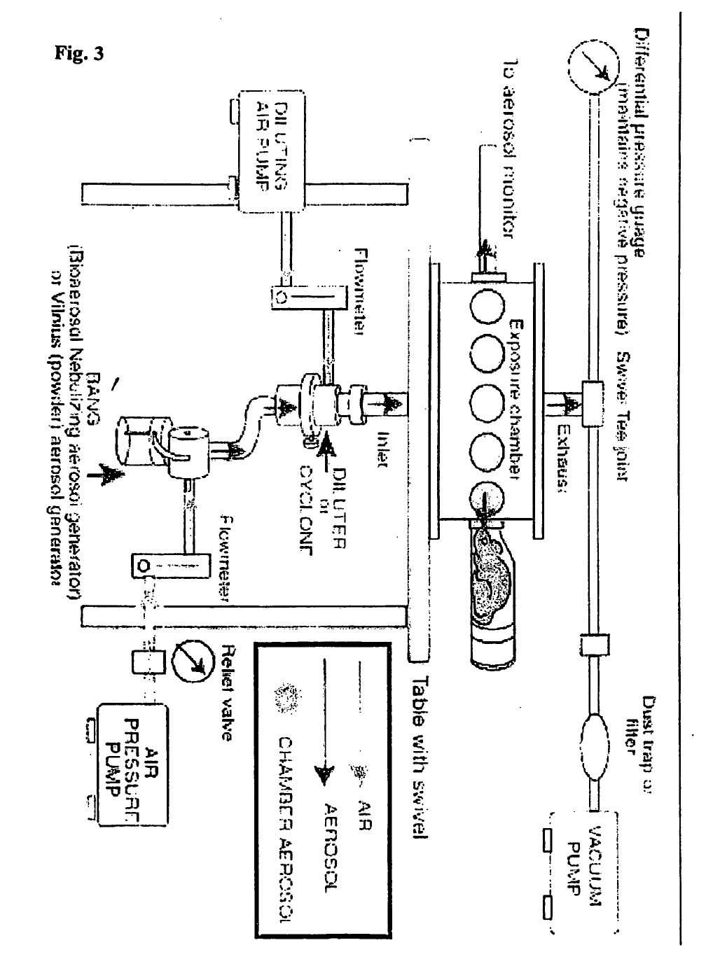

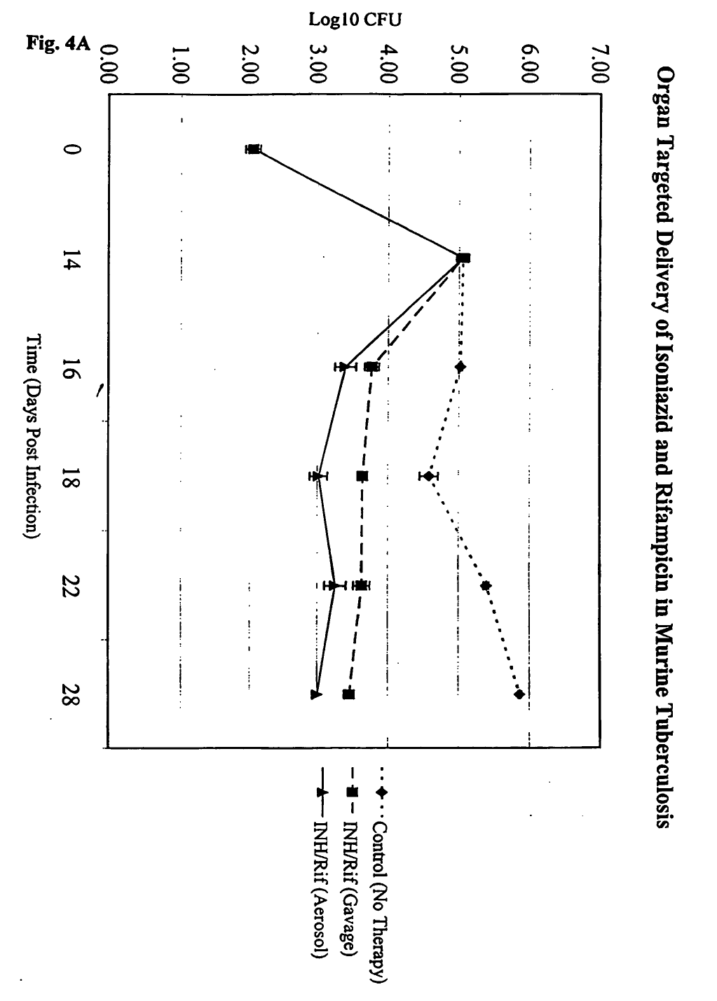

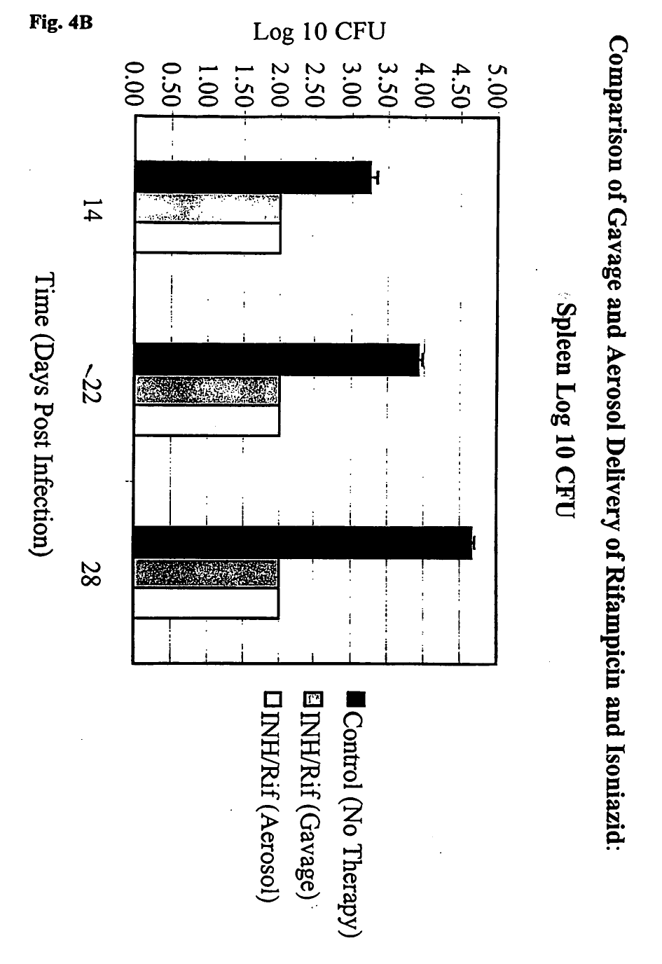

Tuberculosis does not have one in vitro physiologic state that is representative of in vivo metabolic activity. The primary difficulty in the six month standard “short course” chemotherapy is related to the difficulty in eradicating the “persistor” phenotype of M. tuberculosis sporadically replicates in the intracellular environment of the host macrophage. Activity against organisms in this state is often referred to as “sterilizing activity” and is directly correlated with the propensity of patients to relapse, or fail, the six month course of antibiotics. In reviewing the human trials of the British Medical Research Council, rifampicin seemed to have the greatest sterilizing activity and therefore serves as the benchmark of comparison in any in vitro evaluation. Many models have been evaluated seeking in vitro drug evaluations that may best serve to screen for potential utility in in vivo experiments. These include oxygen-deprived cultures, nutrient starved cultures, or simple stationary-phase cultures. Using rifampicin as a drug of comparison for in vitro screening against tuberculosis in conditions other than log phase replicative growth can serve as an indicator of potential benefit in vivo against the persistor phenotype. Stationary phase growth was chosen to provide an evaluation of the activity of capreomycin in comparison to rifampicin. The MTB strain H37Rv, engineered to contain the firefly luciferase gene as a reporter, was allowed to reach stationary phase growth for a minimum of 2 weeks after which the cultures were diluted to an OD650 of 0.07, at which time either rifarnpin or capreomycin was added to the broth culture at 10 times the MIC. Relative light units (“RLUs”) were then assayed and CFUs plated for correlation (FIGS. 2A and B).

Evaluating efficacy in stationary phase cultures revealed that after 18-24 hours of incubation, isoniazid had no significant effect on RLU reduction when compared with log phase growth. After 18-24 hours, rifampicin reduced RLUs by 0.3 logs in stationary phase yet at the same time point, in log phase growth, reduced RLUs by 2.25. Capreomycin maintained the same log reduction in both environments, approximately 2 log decrease in RLUs during stationary and log phase growth at 24 hrs., an approximate 15% reduction in efficacy from log phase cultures. This efficacy was confirmed by 7H11 plating and enumeration of CFUs (FIG. 2B). In evaluating CFUs, capreomycin at day 1 of culture reduces CFUs by 2.5 logs whereas rifampicin has a 0.5 log decrease. The margin of difference decreases over time, presumable as a result of physiologic change of the mycobacteria as they enter log phase growth after the dilution, which would then increase their susceptibility to the profiles described in FIG. 1. As shown in FIGS. 2A and B, isoniazid has no effect at 24 hrs and begins to have an effect only at 48 hrs, potentially related once again to the metabolic variation in the bacillus.

### Example 2

This Example sets forth the results of in vivo studies of the effect of aerosolized capreomycin.

To study the effect of aerosolized capreomycin, a murine model of aerosol drug delivery was developed using a Bioaerosol Nebulizing Generator (BANG) by CH Technologies (USA) Inc. (Westwood, N.J.). The aerosol parameters of the BANG reveals a Mass Median Aerodynamic Diameter of 1.633 microns and a respirable fraction of 86.9% (The portion estimated to deposit in the bronchial and alveolar regions of the pulmonary tree).

The BANG was utilized for the aerogenic infection of C57/BL6 mice with the reference MTB strain H37Rv. Time point zero controls confirmed the deposition of approximately 100 bacilli per mouse. After two weeks of allowing the infection to progress, the mice were divided into 3 groups of 3-4 mice. Group I received no therapy, group 2 received isoniazid and rifampicin by gavage, and group 3 received isoniazid and rifampicin by aerosol (liposomal rifampicin supplied by RT Dodge & Co., Dayton, Ohio). Mice were evaluated at 0, 2, 4, 8, and 14 days after therapy initiation (that is, at 14, 16, 18, 22, and 28 days post infection).

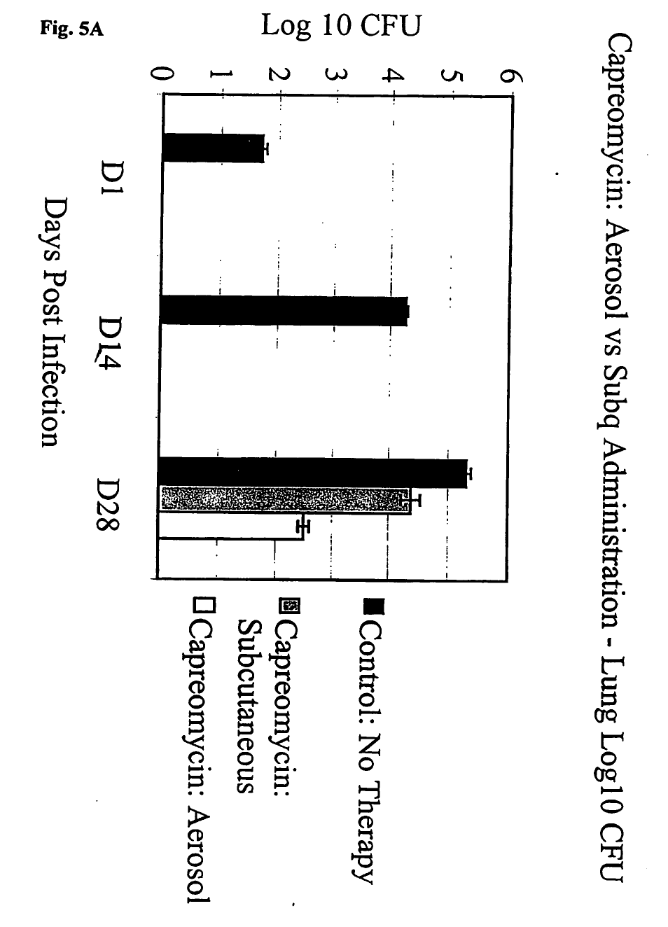

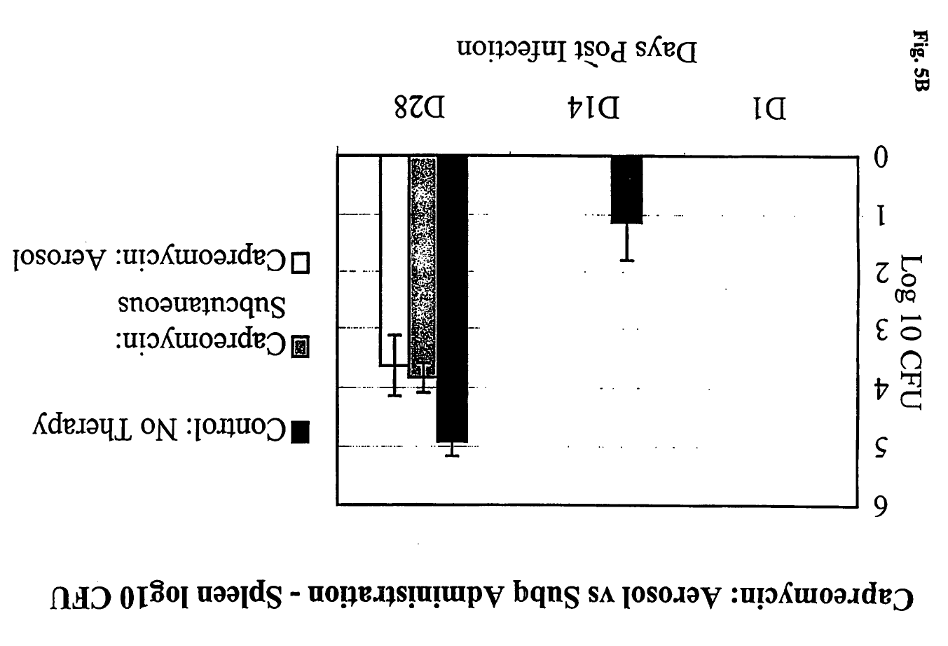

Controlling for the pharmacokinetics of the drugs administered, aerosol administration appeared to improve efficacy of the drugs in the lungs but to show no difference in clearance from the spleen (lower limit of detection log 2). Capreomycin had similar efficacy in aerosol formulation but had decreased efficacy compared with subcutaneous administration in the spleen (FIG. 5). These studies were not designed to specifically evaluate end organ toxicities such as liver enzyme abnormalities or changes in creatinine clearance. A surrogate of systemic drug concentration would be efficacy of MTB elimination in the spleen, which was not as robust as subcutaneous administration. The implication would be that local pulmonary eradication can occur without necessarily exposing the host to the same drug concentration and potentially minimizing systemic toxicities.

More specifically, the reduction in lung CFU at 14 days post therapy initiation (28 days post infection) approaches 2.5 logs in the aerosol treated group whereas the subcutaneous administered group achieved a 1 log reduction at the same time point. More importantly, there was no apparent difference in spleen CFU reduction, implying that the drug concentration in the lungs when administered by aerosol administration is greater than that absorbed when administered systemically.

We have found that specific antimycobacterial agents have variable efficacies depending on how long the mouse has been infected—the more chronic the infection the greater drug tolerance is induced in the host. Chronically infected mice are intuitively closer to a presenting tuberculosis patient than are mice infected for short intervals. Human patients often wait until gross symptoms (such as weight loss and hemoptysis) are apparent, and typically have bacilli in their sputum, indicating a heavy burden of organism. The similarities between latent murine TB and human clinical disease are important since bacilli within infected patients often display phenotypic drug tolerance, reflective of metabolic variations, that makes the organism difficult to eradicate even with drugs, such as isoniazid, that clearly demonstrate efficacy against logarithmically growing organisms. Isoniazid, which is able to reduce the lung CFU by 2 logs two weeks post infection, loses this efficacy by 13 weeks post infection, reducing the bacillary load in the lungs by only 0.5 logs.

Rifampicin maintains the greatest efficacy in this model as can be evidence by the slower decrease in efficacy over time. Since rifampicin is believed to be the most potent sterilizing agent available, effective against the persistor phenotype, this indicates that drugs that are shown to be effective in our chronic murine model mirror efficacy in human hosts.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A system comprising capreomycin and a device for introducing said capreomycin into gases for inhalation by a person in need thereof.

2. A system of claim 1, wherein said capreomycin is introduced into said gases as a solution, a suspension, a powder, or a spray.

3. A system of claim 1, wherein said device is a nebulizer, a metered dose inhaler, or a dry powder inhaler.

4. A system of claim 3, wherein said nebulizer is selected from the group consisting of a heated nebulizer, an ultrasonic nebulizer, a gas nebulizer, a venturi nebulizer, and a refillable nebulizer.

5. A system of claim 1, wherein said capreomycin is introduced into said gases in an average particle size of between 1 and 10 microns.

6. A system of claim 5, wherein said capreomycin has an average particle size of between 2 and 6 microns.

7. A system of claim 5, wherein said capreomycin has an average particle size of about 3 to about 5 microns.

8. A system of claim 1, wherein said capreomycin is provided as a powder.

9. A system of claim 8, wherein said capreomycin is introduced into said gases in an average particle size of between 1 and 10 microns.

10. A system of claim 9, wherein said capreomycin has an average particle size of about 3 to about 5 microns.

11. A formulation of capreomycin suitable for aerosol administration.

12. A formulation of capreomycin of claim 11, wherein said capreomycin has an average particle size between 1 and 10 microns.

13. A formulation of capreomycin of claim 12, wherein said capreomycin is complexed or associated with a polysaccharide.

14. A method of inhibiting the growth of Mycobacterium tuberculosis (“MTB”), said method comprising introducing capreomycin into gases to be inhaled by a patient in need thereof.

15. A method of claim 14, wherein said capreomycin is introduced into said gases as a solution, a suspension, a powder, or a spray.

16. A method of claim 14, wherein said capreomycin introduced into said gases in an average particle size of between 1 and 10 microns.

17. A method of claim 14, wherein said capreomycin is complexed or associated with a polysaccharide.

18. A method of claim 14, wherein said capreomycin is introduced into said gases by a nebulizer, a metered dose inhaler, or a dry powder inhaler.

19. A method of claim 18, wherein said nebulizer is selected from the group consisting of a heated nebulizer, an ultrasonic nebulizer, a gas nebulizer, a venturi nebulizer, and a refillable nebulizer.

20. A method of inhibiting the growth of Mycobacterium tuberculosis (“MTB”) in a patient, said method comprising administering to a lung of said patient aerosolized capreomycin, wherein said capreomycin inhibits the growth of MTB in said patient.

21. A method of claim 20, wherein said capreomycin is administered to said lung as a solution, a suspension, a powder, or a spray.

22. A method of claim 20, wherein said capreomycin is administered to said lung by a nebulizer, a metered dose inhaler, or a dry powder inhaler.

23. A method of claim 22, wherein said nebulizer is selected from the group consisting of a heated nebulizer, an ultrasonic nebulizer, a gas nebulizer, a venturi nebulizer, and a refillable nebulizer.

24. A method of reducing infectivity of a person infected with Mycobacterium tuberculosis (“MTB”), said method comprising administering to the lung of said person aerosolized capreomycin, wherein said capreomycin reduces the infectivity of said person.

25. A method of claim 24, wherein said capreomycin is administered to said lung as a solution, a suspension, a powder, or a spray.

26. A method of claim 24, wherein said capreomycin is administered to said lung by a nebulizer, a metered dose inhaler, or a dry powder inhaler.

27. A method of claim 26, wherein said nebulizer is selected from the group consisting of a heated nebulizer, an ultrasonic nebulizer, a gas nebulizer, a venturi nebulizer, and a refillable nebulizer.

28. A use of capreomycin for manufacture of a medicament for aerosolized administration to a lung as a solution, a suspension, a powder, or a spray.

29. A use of claim 28, wherein said medicament is suitable for delivery to said lung by a nebulizer, a metered dose inhaler, or a dry powder inhaler.

30. A formulation of lyophilized capreomycin having an average particle size of from about 1 to about 10 microns.
